(19) United States
(12) Patent Application Publication
(10) Pub. No.: US 2024/0341693 A1
(43) Pub. Date: Oct. 17, 2024

(54) SYSTEM AND METHODS FOR NON-RESPONSIVE SENSOR DETECTION

(71) Applicant: Dexcom, Inc., San Diego, CA (US)

(72) Inventors: Qi AN, San Diego, CA (US); Kevin CHENG, San Diego, CA (US); Anna C. HARLEY-TROCHIMCZYK, San Diego, CA (US); Avid NAJDAHMADI, San Diego, CA (US); Joshua Ray WINDMILLER, San Diego, CA (US); Hadley Faith VANRENTERGHEM, San Francisco, CA (US); Mohamed R. HELAYHEL, San Diego, CA (US); Peter C. SIMPSON, San Diego, CA (US)

(21) Appl. No.: 18/627,989

(22) Filed: Apr. 5, 2024

Related U.S. Application Data
(60) Provisional application No. 63/496,014, filed on Apr. 13, 2023.

(51) Int. Cl. A61B 5/00; A61B 5/145

(52) U.S. Cl. CPC A61B 5/7221; A61B 5/0004; A61B 5/14532; A61B 5/14546; A61B 5/7203

(57) ABSTRACT

The present disclosure relates to methods and systems for predicting glycemic events in a patient induced as a result of physical activity. In certain aspects, a method includes monitoring a plurality of analytes of the patient continuously during a time period to obtain analyte signals, the plurality of analytes including at least glucose and lactate. The method further includes processing the analyte data from the time period to determine signal-to-noise ratios of the analyte signal. The method then compares the signal-to-noise ratios to each other to determine if a sensor in the continuous analyte monitoring system is non-responsive. In alternative embodiments, a reference signal is obtained and a reference signal-to-noise ratio is compared to the analyte signal-to-noise ratio to determine if a sensor in the continuous analyte monitoring system is non-responsive.

### CROSS-REFERENCE TO RELATED APPLICATIONS

This application claims priority to U.S. Provisional Patent Application No. 63/496,014, filed Apr. 13, 2023, which is hereby assigned to the assignee hereof and hereby expressly incorporated by reference herein in its entirety as if fully set forth below and for all applicable purposes.

### BACKGROUND

Diabetes mellitus is a metabolic condition relating to the production or use of insulin by the body. Insulin is a hormone that allows the body to use glucose for energy, or store glucose as fat.

When a person cats a meal that contains carbohydrates, the digestive system absorbs nutrients, ultimately depositing glucose in the person's blood. Blood glucose can be used for energy or stored as fat. The body normally maintains blood glucose levels in a range that provides sufficient energy to support bodily functions and avoids problems that can arise when glucose levels are too high, or too low. Regulation of blood glucose levels depends on the production and use of insulin, which regulates the movement of blood glucose into cells.

When the body does not produce enough insulin, or when the body is unable to effectively use insulin that is present, blood sugar levels can elevate beyond normal ranges. The state of having a higher than normal blood sugar level is called “hyperglycemia.” Chronic hyperglycemia can lead to a number of health problems, such as cardiovascular disease, cataract and other eye problems, nerve damage (neuropathy), skin ulcers, and kidney damage. Hyperglycemia can also lead to acute problems, such as diabetic ketoacidosis-a state in which the body becomes excessively acidic due to the production of excess ketones, or body acids. The state of having lower than normal blood glucose levels is called “hypoglycemia.” Severe hypoglycemia can lead to damage of the heart muscle, neurocognitive dysfunction, and in certain cases, acute crises that can result in seizures or even death.

A patient living with diabetes can receive insulin to manage blood glucose levels. Insulin can be received, for example, through a manual injection with a needle. Wearable insulin pumps are also available. Diet and exercise also affect blood glucose levels.

Diabetes conditions are sometimes referred to as “Type 1” and “Type 2”. A Type 1 diabetes patient is typically able to use insulin when it is present, but the body is unable to produce sufficient amounts of insulin, because of a problem with the insulin-producing beta cells of the pancreas. A Type 2 diabetes patient may produce some insulin, but the patient has become “insulin resistant” due to a reduced sensitivity to insulin. The result is that even though insulin is present in the body, the insulin is not sufficiently used by the patient's body to effectively regulate blood sugar levels.

Patients with diabetes can benefit from real-time diabetes management guidance, as determined based on a physiological state of the patient, in order to stay within a target glucose range and avoid physical complications. In certain cases, the physiological state of the patient is determined using monitoring systems that measure glucose levels, which inform the identification and/or prediction of adverse glycemic events, such as hyperglycemia and hypoglycemia, and the type of guidance provided to the patient.

For example, such monitoring systems may utilize a continuous glucose monitor (CGM) to measure a patient's glucose levels over time. The measured glucose levels may then be processed by the monitoring system to identify and/or predict adverse glycemic events, and/or to provide guidance to the patient for treatment and or actions to abate or prevent the occurrence of such adverse glycemic events. For example, trends, statistics, or other metrics may be derived from the glucose levels and used to identify and/or predict adverse glycemic events. Or, in certain cases, the glucose levels themselves may be used to identify and/or predict adverse glycemic events.

Even with the systems described above, however, the management of diabetes presents many challenges for patients, clinicians, and caregivers, as a confluence of various factors can impact a patient's glucose levels, thus affecting the accuracy of glycemic event prediction and the guidance provided by diagnostics systems.

### SUMMARY

Certain embodiments of the present disclosure describe a continuous analyte monitoring system that uses signal processing to determine when an analyte sensor has become defective. According to an embodiment, an analyte sensor system includes a sensor, a memory, and a processor communicatively coupled to the memory. The processor generates, using the sensor, a reference analyte signal stream and determines a reference signal noise in the reference analyte signal stream. The processor also compares the reference analyte signal stream to the reference signal noise to determine a reference signal-to-noise ratio and generates, using the sensor, an analyte signal stream. The processor further determines a signal noise in the analyte signal stream, determines a signal-to-noise ratio based on the analyte signal stream and the signal noise, and in response to determining that the signal-to-noise ratio is less than the reference signal-to-noise ratio, determines that the sensor is defective.

According to another embodiment, an analyte sensor system includes a first sensor, a second sensor, a memory, and a processor communicatively coupled to the memory. The processor generates, using the first sensor, a first analyte signal stream indicating a level of a first analyte and determines a first signal noise in the first analyte signal stream. The processor also determines a first signal-to-noise ratio based on the first analyte signal stream and the first signal noise and generates, using the second sensor, a second analyte signal stream indicating a level of a second analyte. The processor further determines a second signal noise in the second analyte signal stream, determines a second signal-to-noise ratio based on the second analyte signal stream and the second signal noise, and in response to determining that the second signal-to-noise ratio exceeds the first signal-to-noise ratio, determines that the first sensor is defective.

According to another embodiment, a method includes generating, using a sensor, a reference analyte signal stream and determining a reference signal noise in the reference analyte signal stream. The method also includes comparing the reference analyte signal stream to the reference signal noise to determine a reference signal-to-noise ratio and generating, using the sensor, an analyte signal stream. The method further includes determining a signal noise in the analyte signal stream, determining a signal-to-noise ratio based on the analyte signal stream and the signal noise, and in response to determining that the reference signal-to-noise ratio exceeds the signal-to-noise ratio, determining that the sensor is defective.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only exemplary embodiments and are therefore not to be considered limiting of its scope, may admit to other equally effective embodiments.

FIG. 1 illustrates aspects of an example disease management system used in connection with implementing embodiments of the present disclosure.

FIG. 2 is a diagram conceptually illustrating an example continuous analyte monitoring system including example continuous analyte sensors with sensor electronics, in accordance with certain aspects of the present disclosure.

FIG. 3 is a block diagram that illustrates electronics associated with the sensor system of FIGS. 1 and 2, according to certain embodiments disclosed herein.

FIG. 4A illustrates a flow diagram depicting an example method for determining that an analyte sensor is defective, according to some embodiments disclosed herein.

FIG. 4B illustrates a flow diagram depicting an example method for determining that an analyte sensor is defective, according to some embodiments disclosed herein.

FIGS. 5A and 5B illustrate analyte signals and noise signals of an analyte sensor, according to certain aspects of the present disclosure.

To facilitate understanding, identical reference numerals have been used, where possible, to designate identical elements that are common to the figures. It is contemplated that elements and features of one embodiment may be beneficially incorporated in other embodiments without further recitation.

### DETAILED DESCRIPTION

In a continuous glucose monitoring system, a transcutaneous, subcutaneous, or intracutaneous continuous glucose sensor that is inserted into the interstitial fluid is used to monitor a patient's glucose levels, thereby, providing glucose measurements reflective of the physiological state of the patient. However, monitoring multiple analytes, in addition to glucose, may provide a more complete picture of the physiological state of the patient. Examples of such analytes include, ketones, lactate, insulin, electrolytes, creatinine, as well as a number of other biomarkers including proteins, metabolites, ions, vitamins, minerals, hormones, and nucleic acids. Analytes can also include therapeutic agents or other exogenous compounds such as anti-biotic agents, anti-viral agents, acetaminophen, metformin, statins, GLP-1 receptor agonists, insulin, and SGLT-2 inhibitors.

Over time, a transcutaneous, subcutaneous, or intracutaneous multi-analyte sensor that, for example, monitors one or more of the analytes above, may become defective or non-responsive. The sensor may be physically damaged, for example, from insertion or other impact (e.g., user movement) that causes the sensor to not function as intended. The sensor may also be come inaccurate or exhibit signal-to-noise erosion due to several factors, such as pressure-induced sensor artifacts, fluctuating temperatures at the sensor or biological interface, increased biofouling or foreign body reaction at the sensor or biological interface (e.g., encapsulation, foreign body giant cell formation, and/or collagen formation), progressive sensitivity decline, reference electrode instability or loss of reference capacity, and/or membrane evolution in situ. A defective sensor may generate analyte signal streams with lower amplitudes. Additionally, the defective sensor may produce excessive noise that interferes with or obscures the analyte signal streams generated by the defective sensor. For example, the noise incident on the signal (which may also be referred to as “signal noise”) may mask or even exceed the amplitude of the analyte signal streams generated by the sensor, leading to inaccurate sensor readings. These inaccurate sensor readings may then indicate inaccurate analyte level measurements and, therefore, false glycemic events (e.g., hyper or hypoglycemic events), interfere with analyte trend or pattern analyses, and/or lead to inaccurate insulin dosage calculations and delivery.

The present disclosure describes a continuous analyte monitoring system that uses signal processing to determine when an analyte sensor has become defective. For example, first, the continuous analyte monitoring system determines a signal noise present in an analyte signal stream produced by an analyte sensor. The continuous analyte monitoring system may then also determine a signal-to-noise ratio of the analyte signal stream using the signal noise. The continuous analyte monitoring system may next compare the signal-to-noise ratio with a reference signal-to-noise ratio or a signal-to-noise ratio of another analyte signal stream generated by another analyte sensor to determine whether the analyte sensor is defective. For example, if the signal-to-noise ratio of the analyte signal stream exceeds the reference signal-to-noise ratio or the signal-to-noise ratio of the other analyte signal stream, then the continuous analyte monitoring system may determine that the analyte sensor is not defective. If the reference signal-to-noise ratio or the signal-to-noise ratio of the other analyte signal stream exceed the signal-to-noise ratio of the analyte signal stream, then the continuous analyte monitoring system may determine that the analyte sensor is defective. The continuous analyte monitoring system may then communicate (e.g., to a display device) a notification, alert, alarm, or other visual, audible, or haptic indication that the analyte sensor is defective and should be replaced. The reference signal-to-noise ratio may be determined according to any measurements (e.g., a sensor current measurement, a sensor voltage measurement, a sensor impedance measurement, a sensor resistance measurement, a sensor capacitance measurement, and/or a sensor transconductance measurement). The reference signal-to-noise ratio may be determined using a single measurement or multiple measurements (e.g., a time-series dataset).

The continuous analyte monitoring system may monitor the reference signal-to-noise ratio to determine whether the other analyte sensor is defective. For example, if the reference signal-to-noise ratio falls below a threshold (which may be referred to as signal-to-noise erosion), the continuous analyte monitoring system may determine that the other analyte sensor is defective and should be replaced. The continuous analyte monitoring system may communicate a notification, alert, alarm, or other visual, audible, or haptic indication that the other analyte sensor is defective and should be replaced. In certain embodiments, when the reference signal-to-noise ratio falls below the threshold, the continuous analyte monitoring system may determine that both the analyte sensor and the other analyte sensor are defective and should be replaced. In another embodiment, the continuous analyte monitoring system may invoke an algorithmic routine intended to compensate for said erosion of the signal-to-noise ratio. In yet another embodiment, the continuous analyte monitoring system may shift to measurement of another, independent analyte sensor contained within the continuous analyte monitoring system. The continuous analyte monitoring system may indicate through the notification, alert, alarm, or other indication that both analyte sensors should be replaced.

In some embodiments, the continuous analyte monitoring system includes a sensor that may not be an analyte sensor (e.g., the sensor may lack an enzyme used to detect an analyte). The continuous analyte monitoring system may use this sensor to measure background noise or a reference signal. The continuous analyte monitoring system may use the background noise or a reference signal detected by this sensor as the signal noise used to determine the signal-to-noise ratio for the analyte signal stream produced by the analyte sensor and/or the reference signal-to-noise ratio. The noise is an additive quantity and can be physiologic in origin (e.g., sensor encapsulation via the foreign body response) in addition to having physical contributions (e.g., Brownian noise, thermal noise, shot noise, flicker noise, etc.).

In certain embodiments, comparing the signal-to-noise ratio of the analyte signal stream to the signal-to-noise ratio of the other analyte signal stream or the reference signal-to-noise ratio allows for identification of a defective analyte sensor, which may lead to the defective analyte sensor being replaced with an analyte sensor that produces more accurate analyte measurements.

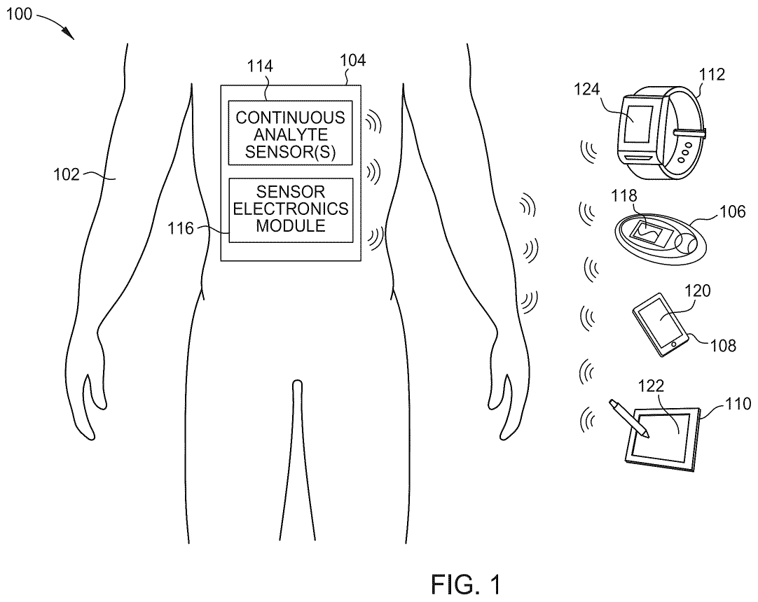

FIG. 1 illustrates an example system 100, including a continuous analyte monitoring system 104, and display devices 106, 108, 110, and 112. The system 100 may continuously monitor one or more analytes of the user 102, in accordance with certain aspects of the present disclosure. The continuous analyte monitoring system 104 includes one or more continuous analyte sensors 114 and a sensor electronics module 116. The sensor electronics module 116 may be in wired or wireless communication (e.g., directly or indirectly) with one or more of the display devices 106, 108, 110, and 112.

A continuous analyte sensor 114 may include one or more analyte sensors for measuring analytes. A continuous analyte sensor 114 may include a multi-analyte sensor that continuously measures two or more analytes (e.g., glucose, lactate, uric acid, sodium, potassium, ketone, creatinine, etc.), and/or multiple single analyte sensors, each continuously measuring a single analyte (e.g., where one continuous analyte sensor 114 is used for measuring glucose and then a second continuous analyte sensor 114 used for measuring lactate, etc.). The continuous analyte sensor 114 may be a non-invasive device, a minimally-invasive device, a skin-adhered device, a subcutaneous device, a transcutaneous device, a subdermal device, an intradermal device, a transdermal device, or an intravascular device. The continuous analyte sensor 114 may continuously measure analyte levels of a user using one or more techniques, such as enzymatic techniques, ion-selective techniques, aptameric techniques, chemical techniques, physical techniques, electrochemical techniques, spectrophotometric techniques, polarimetric techniques, calorimetric techniques, iontophoretic techniques, radiometric techniques, immunochemical techniques, and the like. The continuous analyte sensor 114 may provide a signal stream indicative of a level (e.g., a concentration) of one or more analytes in the user over time. The signal stream may vary over time as the level of the one or more analytes changes over time.

An analyte may be a substance or chemical constituent in a biological fluid (for example, blood, interstitial fluid, cerebral spinal fluid, lymph fluid, sweat, or urine) that can be analyzed. Analytes can include naturally occurring substances, endogenous substances, exogenous substances, artificial substances, pharmacologic agents, metabolites, electrolytes, ions, blood gasses, minerals, vitamins, proteins, enzymes, or reaction products. Analytes for measurement by the devices and methods may include, but may not be limited to, glucose, acarboxyprothrombin; acylcarnitine; adenine phosphoribosyl transferase; adenosine deaminase; albumin; alpha-fetoprotein; amino acid profiles (arginine (Krebs cycle), histidine/urocanic acid, homocysteine, phenylalanine/tyrosine, tryptophan); andrenostenedione; antipyrine; arabinitol enantiomers; arginase; benzoylecgonine (cocaine); bicarbonate; biotinidase; biopterin; blood urea nitrogen; c-reactive protein; calcium; carbon dioxide; carnitine; carnosinase; CD4; ceruloplasmin; chenodeoxycholic acid; chloride; chloroquine; cholesterol; cholinesterase; conjugated 1-β hydroxy-cholic acid; cortisol; creatine kinase; creatine kinase MM isoenzyme; cyclosporin A; d-penicillamine; de-cthylchloroquine; dehydroepiandrosterone sulfate; DNA (acetylator polymorphism, alcohol dehydrogenase, alpha 1-antitrypsin, cystic fibrosis, Duchenne/Becker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheria/tetanus antitoxin; erythrocyte arginase; erythrocyte protoporphyrin; esterase D; fatty acids/acylglycines; free β-human chorionic gonadotropin; free erythrocyte porphyrin; free thyroxine (FT4); free tri-iodothyronine (FT3); fumarylacetoacetase; galactose/gal-1-phosphate; galactose-1-phosphate uridyltransferase; gentamicin; glucose-6-phosphate dehydrogenase; glutathione; glutathione perioxidase; glycocholic acid; glycerol; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), B/A-1, β); lysozyme; mefloquine; netilmicin; phenobarbitone; phenytoin; phytanic/pristanic acid; potassium; progesterone; prolactin; prolidase; purine nucleoside phosphorylase; potassium, quinine; reverse tri-iodothyronine (rT3); selenium; serum pancreatic lipase; sissomicin; sodium; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measles/mumps/rubella, Mycobacterium leprac, Mycoplasma pneumoniac, Myoglobin, Onchocerca volvulus, oxygen parainfluenza virus, Plasmodium falciparum, poliovirus, Pseudomonas acruginosa, respiratory syncytial virus, rickettsia (scrub typhus), Schistosoma mansoni, Toxoplasma gondii, Trepenoma pallidium, Trypanosoma cruzi/rangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin.

Salts, sugar, protein, fat, vitamins, and hormones naturally occurring in blood or interstitial fluids can also constitute analytes in certain implementations. The analyte can be naturally present in the biological fluid, for example, a metabolic product, a hormone, a neurotransmitter, a signaling agent, an antigen, an antibody, and the like. Alternatively, the analyte can be introduced into the body or exogenous, for example, a contrast agent for imaging, a radioisotope, a chemical agent, a fluorocarbon-based synthetic blood, or a drug or pharmaceutical composition, including but not limited to insulin; glucagon, sodium-glucose co-transporter 2 inhibitors (SGLT-2i), glucagon-like peptide 1 (GLP-1) agonists; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, catecholamines (L-DOPA, dopamine, epinephrine, norepinephrine), methamphetamines, Ritalin, Cylert, Preludin, Didrex, PreState, Voranil, Sandrex, Plegine); depressants (barbiturates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-Dihydroxyphenylacetic acid (DOPAC), Homovanillic acid (HVA), 5-Hydroxytryptamine (5HT), and 5-Hydroxyindoleacetic acid (FHIAA), and intermediaries in the Citric Acid Cycle.

The sensor electronics module 116 includes electronic circuitry for measuring and processing the signal streams from the continuous analyte sensors 114. The sensor electronics module 116 can be physically connected to the continuous analyte sensors 114 and can be integral with (non-releasably attached to) or releasably attachable to the continuous analyte sensors 114. The sensor electronics module 116 may include hardware (including, but not limited to an electrochemical analog front end, microprocessor, battery, and memory), firmware, or software that enable measurement of levels of analytes via the continuous analyte sensors 114. For example, the sensor electronics module 116 can include an electrochemical analog front end (e.g., a potentiostat, controlled voltage device, galvanostat, controlled current device, coulometer, impedance analyzer, frequency response analyzer, etc.), a power source for providing power to the sensor, other components useful for signal processing and data storage, and a telemetry module for transmitting data from the sensor electronics module to, e.g., one or more display devices. Electronics can be affixed to a printed circuit board (PCB), or the like, and can take a variety of forms. For example, the electronics can take the form of an integrated circuit (IC), such as an Application-Specific Integrated Circuit (ASIC), a microcontroller, or a processor. In some embodiments, the sensor electronics module 116 includes a memory and a processor. The memory stores software instructions that are executed by the processor to perform the actions or functions of the continuous analyte monitoring system 104 described herein (e.g., detecting defective continuous analyte sensors 114).

The display devices 106, 108, 110, and 112 may display displayable sensor data, including the detected levels of analytes, which may be transmitted by the sensor electronics module 116. The sensor electronics module 116 may transmit raw sensor data that is converted to displayable sensor data via one or more of the display devices 106, 108, 110, and 112. The sensor electronics module 116 may convert raw sensor data to displayable sensor data and transmit the displayable sensor data to one or more of the display devices 106, 108, 110, and 112. Each of the display devices 106, 108, 110, and 112 may include a display such as a touchscreen display 118, 120, 122, and 124 for displaying sensor data to a user or for receiving inputs from the user. For example, a graphical user interface (GUI) may be presented to the user for such purposes. The display devices 106, 108, 110, and 112 may include other types of user interfaces such as a voice user interface instead of, or in addition to, a touchscreen display for communicating sensor data to the user of the display device or for receiving user inputs. The display devices 106, 108, 110, and 112 may display or otherwise communicate the sensor data as it is communicated from the sensor electronics module 116 (e.g., in a customized data package that is transmitted to the display devices 106, 108, 110, and 112 based on their respective preferences).

The display devices 106 may include a custom display device specially designed for displaying certain types of displayable sensor data for analyte data received from the sensor electronics module 116. The display device 108 may be a smartphone or a mobile phone using a commercially available operating system (OS) and may display a graphical representation of the continuous sensor data (e.g., including current and historic data). The display device 110 may include a tablet, and the display device 112 may include a smart watch. The display devices 106, 108, 110, and 112 may include a desktop or laptop computer (not shown).

Because different display devices provide different user interfaces, content of the data packages (e.g., amount, format, or type of data to be displayed, alarms, and the like) can be customized (e.g., programmed differently by the manufacture or by an end user) for each particular display device. Accordingly, different display devices can be in direct wireless communication with the sensor electronics module 116 (e.g., such as an on-skin sensor electronics module 116 that is physically connected to continuous analyte sensors 114) during a sensor session to enable a plurality of different types or levels of display or functionality for the displayable sensor information.

The continuous analyte monitoring system 104 or the display devices 106, 108, 110, and 112 may communicate together wirelessly using one of a variety of wireless communication technologies (e.g., Wi-Fi, Bluetooth, Near Field Communication (NFC), NB-IoT, LTE Cat M1, 4G, LTE, 5G, 6G, cellular, etc.). A wireless access point (WAP) may be used to couple one or more of the continuous analyte monitoring system 104 or the display devices 106, 108, 110, and 112 to one another. For example, the WAP may provide Wi-Fi, Bluetooth, or cellular connectivity among these devices. NFC may also be used among the devices in the system 100.

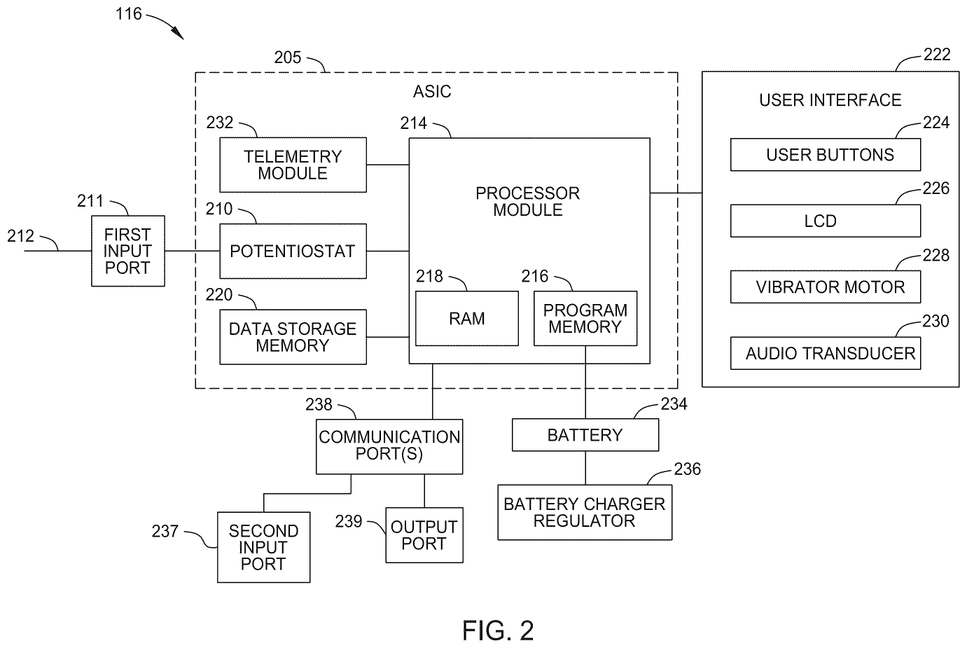

FIG. 2 depicts an example of sensor electronics module 116, in accordance with some example implementations. The sensor electronics module 116 may include sensor electronics that process sensor information, such as sensor data, and generate transformed sensor data and displayable sensor information, e.g., via a processor module 214. For example, the processor module 214 may transform sensor data into one or more of the following: filtered sensor data (e.g., one or more filtered analyte concentration values), raw sensor data, calibrated sensor data (e.g., one or more calibrated analyte concentration values), rate of change information, trend information, rate of acceleration/deceleration information, sensor diagnostic information, analyte ratio information, location information, alarm/alert information, sensor temperature information, sensor impedance information, local oxygen level information, calibration information such as may be determined by factory calibration algorithms as disclosed herein, smoothing or filtering algorithms of sensor data, or the like.

The processor module 214 performs a substantial portion, if not all, of the data processing, including data processing pertaining to factory calibration. The processor module 214 may be integral to the sensor electronics module 204 or may be located remotely. The processor module 214 may include smaller subcomponents or submodules. For example, the processor module 214 may include an alert module (not shown) or prediction module (not shown), or any other suitable module that may be utilized to efficiently process data. When the processor module 214 includes submodules, the submodules may be located within the processor module 214, including within the sensor electronics module 204 or other associated devices. For example, the processor module 214 may be located at least partially within a cloud-based analyte processor.

The sensor electronics module 204 may include an application-specific integrated circuit (ASIC) 205 coupled to a user interface 222. The ASIC 205 may be coupled to the user interface 222 through a wireless connection, e.g., Bluetooth or NFC. The ASIC 205 may further include an electrochemical analog front end, such as potentiostat 210, a telemetry module 232 for transmitting data from the sensor electronics module 204 to one or more devices, such as devices 106, 108, 110, and 112, or other components for signal processing and data storage (e.g., processor module 214 and data storage memory 220). Although FIG. 2 depicts ASIC 205, other types of circuitry may be used as well, including field programmable gate arrays (FPGA), one or more microprocessors that provide some (if not all of) the processing performed by the sensor electronics module 204, analog circuitry, digital circuitry, or a combination thereof.

In the example of FIG. 2, through a first input port 211 for sensor data the potentiostat 210 is coupled to a continuous analyte sensor 114, such as a glucose sensor, to generate sensor data from the analyte. The potentiostat 210 may also provide via data line 212 a voltage to the continuous analyte sensor 114 to bias the sensor for measurement of a value (e.g., a current and the like) indicative of the analyte concentration in a host (also referred to as the analog portion of the sensor). The potentiostat 210 may have one or more channels depending on the number of working electrodes at the continuous analyte sensor 114.

The potentiostat 210 may include a transimpedance amplifier that translates a current value from the continuous analyte sensor 114 into a voltage value, or a current-to-frequency converter (not shown), which may also continuously integrate a measured current value from the continuous analyte sensor 114 using, for example, a charge-counting device. An analog-to-digital converter (not shown) may digitize the analog signal from the continuous analyte sensor 114 into so-called “counts” to allow processing by the processor module 214. The resulting counts may be directly related to the current measured by the potentiostat 210, which may be directly related to an analyte level, such as a glucose level, in the host.

The telemetry module 232 may be operably connected to the processor module 214 and may provide the hardware, firmware, or software that enable wireless communication between the sensor electronics module 116 and one or more other devices, such as display devices, processors, network access devices, and the like. A variety of wireless radio technologies that can be implemented in the telemetry module 232 include Bluetooth, Bluetooth Low-Energy, ANT, ANT+, ZigBee, Thread, IEEE 802.11, IEEE 802.16, cellular radio access technologies (NB-IoT, LTE Cat M1, 4G, LTE, 5G, 5G NR, 6G), radio frequency (RF), infrared (IR), paging network communication, magnetic induction, satellite data communication, spread spectrum communication, frequency hopping communication, near field communications (NFC), or the like. The telemetry module 232 may include a Bluetooth chip, although Bluetooth technology may also be implemented in a combination of the telemetry module 232 and the processor module 214.

The processor module 214 may control the processing performed by the sensor electronics module 116. For example, the processor module 214 may process data (e.g., counts), from the sensor, filter the data, calibrate the data, normalize the data, smooth the data, perform fail-safe checking, or the like. The processor module 214 may include a processor and a memory that perform the functions or actions of the processor module 214.

The processor module 214 may include a digital filter, such as a high-pass filter, a low-pass filter, a band-pass filter, a notch filter, an infinite impulse response (IIR), a finite impulse response (FIR) filter, a sinc filter, a Bessel filter, a bilinear transform, a Butterworth filter, a Chebyshev filter, a Savitzky-Golay filter, an elliptical filter, an adaptive filter, or a Kalman filter. This digital filter may smooth a raw data stream received from the continuous analyte sensor 114. Generally, the digital filters are programmed to filter data sampled at a predetermined time interval (also referred to as a sample rate). When the potentiostat 210 measures the analyte (e.g., glucose or the like) at discrete time intervals, these time intervals determine the sampling rate of the digital filter. The potentiostat 210 may measure continuously the analyte, for example, using a current-to-frequency converter. In these current-to-frequency converter implementations, the processor module 214 may be programmed to request, at predetermined time intervals (acquisition time), digital values from the integrator of the current-to-frequency converter. These time intervals may change dynamically or adaptively depending on a rate of change, inflection, or absolute value of a detected analyte level. These digital values obtained by the processor module 214 from the integrator may be averaged over the acquisition time due to the continuity of the current measurement. As such, the acquisition time may be determined by the sampling rate of the digital filter.

The processor module 214 may further include a data generator (not shown) that generates data packages for transmission to devices, such as the display devices 106, 108, 110, and 112. Furthermore, the processor module 214 may generate data packets for transmission to these outside sources via the telemetry module 232. In some example implementations, the data packages may, as noted, be customizable for each display device, or may include any available data, such as a time stamp, displayable sensor information, transformed sensor data, an identifier code for the sensor or sensor electronics module 116, raw data, filtered data, calibrated data, rate of change information, trend information, error detection or correction, temperature information, impedance information, local oxygen level information, kinesthetic information, or the like.

The processor module 214 may also include a program memory 216 and other memory 218. The processor module 214 may include a processor that executes software instructions stored in the program memory 216 and the memory 218 to perform the actions or functions of the continuous analyte monitoring system 104 described herein. The processor module 214 may be coupled to a communications interface, such as a communication port 238, and a source of power, such as a battery 234. Moreover, the battery 234 may be further coupled to a battery charger or regulator 236 to provide power to sensor electronics module 116 or charge the battery 234.

The program memory 216 may be implemented as a semi-static memory for storing data, such as an identifier for a coupled continuous analyte sensor 114 (e.g., a sensor identifier (ID)) and for storing code (also referred to as program code) to configure the ASIC 205 to perform one or more of the operations/functions described herein. For example, the program code may configure processor module 214 to process data streams or counts, filter, perform the calibration methods described below, perform fail-safe checking, and the like.

The memory 218 may also be used to store information. For example, the processor module 214 including memory 218 may be used as the system's cache memory, where temporary storage is provided for recent sensor data received from the sensor. In some example implementations, the memory may include memory storage components, such as read-only memory (ROM), random-access memory (RAM), dynamic-RAM, static-RAM, non-static RAM, write-once memory (WOM), one-time-programmable (OTP)_memory, easily erasable programmable read only memory (EEPROM), rewritable ROMs, flash memory, and the like.

The data storage memory 220 may be coupled to the processor module 214 and may store a variety of sensor information. In some example implementations, the data storage memory 220 stores one or more days of continuous analyte sensor data. For example, the data storage memory may store 1, 2, 3, 4, 5, 6, 7, 8, 9, 10, 11, 12, 13, 14, 15, or more days (e.g., 20 days) of continuous analyte sensor data received from the continuous analyte sensor 114. The stored sensor information may include one or more of the following: a time stamp, raw sensor data (one or more raw analyte concentration values), calibrated data, filtered data, transformed sensor data, or any other displayable sensor information, calibration information (e.g., reference BG values or prior calibration information such as from factory calibration), sensor diagnostic information, temperature information, impedance information, local oxygen level information, kinesthetic information, and the like.

The user interface 222 wirelessly coupled to the ASIC 205 may include a variety of interfaces, such as one or more buttons 224, a liquid crystal display (LCD) 226, a vibrator 228, an audio transducer (e.g., speaker) 230, a backlight (not shown), a microphone (not shown), a light source (e.g., LED, not shown), a photodetector (e.g., photodiode, not shown), or the like. The components that include the user interface 222 may provide controls to interact with the user (e.g., the host). One or more buttons 224 may allow, for example, toggle, menu selection, option selection, status selection, yes/no response to on-screen questions, a “turn off” function (e.g., for an alarm), an “acknowledged” function (e.g., for an alarm), a reset, or the like. The LCD 226 may provide the user with, for example, visual data output. The audio transducer 230 (e.g., speaker) may provide audible signals in response to triggering of certain alerts, such as present or predicted hyperglycemic and hypoglycemic conditions. Audible signals may be differentiated by tone, volume, duty cycle, pattern, duration, or the like. The audible signal may be silenced (e.g., acknowledged or turned off) by pressing one or more buttons 224 on the sensor electronics module 116 or by signaling the sensor electronics module 116 using a button or selection on a display device (e.g., key fob, cell phone, or the like). Although audio and vibratory alarms are described with respect to FIG. 2, other alarming mechanisms may be used as well. For example, in some example implementations, a tactile alarm is provided including a poking mechanism that “pokes” or physically contacts the patient in response to one or more alarm conditions.

The battery 234 may be operatively connected to the processor module 214 (and possibly other components of the sensor electronics module 116) and provide the necessary power for the sensor electronics module 116. The battery 234 may be a lithium manganese dioxide battery, however any appropriately sized and powered battery can be used (e.g., AAA, button cell, nickel-cadmium, zinc-carbon, alkaline, lithium, nickel-metal hydride, lithium-ion, zinc-air, zinc-mercury oxide, silver-zinc, silver oxide, or hermetically-sealed). The battery 234 may be primary or rechargeable. Multiple batteries 234 can be used to power the system. In other representative embodiments, the battery may include a solid-state battery, a flexible battery, surface mount battery, a supercapacitor, an ultracapacitor, an electrochemical double layer capacitor, a thermoelectric generator, a photovoltaic cell, a fuel cell, or a biofuel cell. In yet other implementations, the receiver can be transcutaneously powered via an inductive coupling, for example.

A battery charger or regulator 236 may receive energy from an internal or external charger. The battery regulator (or balancer) 236 regulates the recharging process by bleeding off excess charge current to allow all cells or batteries in the sensor electronics module 116 to be fully charged without overcharging other cells or batteries. The battery 234 (or batteries) may be charged via an inductive or wireless charging pad, although any other charging or power mechanism may be used as well.

One or more communication ports 238, also referred to as external connectors, may be provided to allow communication with other devices, for example a PC communication (com) port can be provided to enable communication with systems that are separate from, or integral with, the sensor electronics module 116. The communication port, for example, may include a serial (e.g., universal serial bus or “USB”) communication port, and allow for communicating with another computer system (e.g., PC, personal digital assistant or “PDA,” server, or the like). The sensor electronics module 116 may transmit historical data to a PC or other computing device (e.g., an analyte processor as disclosed herein) for retrospective analysis by a patient or physician. As another example of data transmission, factory information may also be sent to the algorithm from the sensor or from a cloud data source.

The one or more communication ports 238 may further include a second input port 237 in which calibration data may be received, and an output port 239 which may be employed to transmit calibrated data, or data to be calibrated, to a display device (e.g., display device 106, 108, 110, and 112). FIG. 2 illustrates these aspects schematically. It will be understood that the ports may be separated physically, but in alternative implementations a single communication port may provide the functions of both the second input port and the output port.

Although separate data storage and program memories are shown in FIG. 2, a variety of configurations may be used as well. For example, one or more memories may be used to provide storage space to support data processing and storage requirements at sensor electronics module 116.

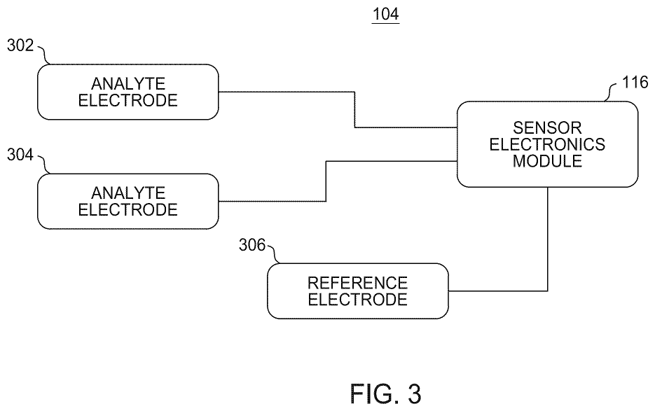

FIG. 3 illustrates an example continuous analyte monitoring systems 104, according to some embodiments disclosed herein. The continuous analyte monitoring system 104 includes a sensor electronics module 116 as well as multiple working electrodes that operate as analyte sensors. As seen in FIG. 3, the continuous analyte monitoring system 104 includes analyte electrodes 302 and 304 (which may also be referred to as “working electrodes”) and a reference electrode 306. Optionally, the continuous analyte monitoring system includes a counter electrode. The continuous analyte monitoring system 104 may include any suitable number of analyte electrodes (e.g., one or more analyte electrodes). Each of the electrodes may include a membrane, or multiple membranes, over its surface. The electrodes may be at least partially covered by the same membrane. Alternatively, one or more of the electrodes may not be covered by a membrane as in a bare electrode. The electrodes may be electrically coupled to the sensor electronics module 116 (e.g., the potentiostat and the processor module of the sensor electronics module 116).

The electrodes may be formed of any suitable materials and by any suitable methods. For example, the electrodes may be formed of one or more noble metals, such as platinum, palladium, rhodium, iridium, ruthenium, or platinum/iridium. In some embodiments, the electrodes are carbon-based, and include carbon, carbon/ruthenium, doped diamond, carbon nanotube, graphene, graphite, amorphous carbon, or carbon fiber. In certain embodiments, the electrodes are formed of graphite, gold, conductive polymer, indium tin oxide, or the like. The reference electrode 308 may include silver, silver/silver chloride, or iridium oxide and may be kept currentless. Generally, suitable methods for forming the electrodes include roll-to-roll techniques, screen printing, slot-die coating, discrete dispense, microfabrication techniques, such as physical vapor deposition, chemical vapor deposition, electrodeposition, lithography, and/or etching techniques. Other methods, including spray deposition, slot-die coating, discrete dispense, foil lamination, or dip-coating, are also contemplated.

The continuous analyte monitoring system 104 may be positioned on a body of a user (e.g., user 102) by inserting some or all of the working electrodes into the body. The working electrodes may be inserted into an adequate insertion site, such as an abdomen or an arm of the user 102, where the working electrodes may be in contact with the blood or interstitial fluid of the user 102.

Different electrodes may detect different analytes. For example, each of the electrodes may include an active surface to facilitate electrochemical sensing of desired analytes. The active surface of each electrode may be voxelated, or partitioned into discrete sections (e.g., cubic sections). Different selective molecular recognition elements (e.g., enzymes, antibodies, aptamers, double-stranded deoxyribonucleic acid (DNA), single-stranded DNA, ribonucleic acid (RNA), oligonucleotides, proteins, cells, microbes, ion-selective materials, etc.), each specific to a different analyte, may be deposited and immobilized on each active surface of each electrode. For example, both glucose oxidase and lactate or uric acid oxidase (and/or other analyte-specific enzymes) may be deposited on the active surface of one electrode. Different selective molecular recognition elements may be immobilized on each voxel of each electrode. In some embodiments, only one type of selective molecular recognition element is deposited on each voxel of each electrodes. The selective molecular recognition elements may be immobilized via adsorption, entrapment, cross-linking, covalent bonding, electrostatic interactions, hydrogen bonding, peptide bonding, thiol-gold bonding, or any other suitable immobilization methods.

Each of electrodes includes selective molecular recognition elements, such as enzymes or ionophores, for one specific analyte, while different electrodes may include selective molecular recognition elements for different analytes. In certain embodiments, some of the electrodes include two or more selective molecular recognition elements, such as four or more selective molecular recognition elements, which together enable sensing of a single analyte. For example, where an electrode is configured to sense creatinine, the electrode may include four or more enzymes specific for creatinine. An example of a 1-enzyme sensor electrode may include a lactate-specific electrode, and an example of a 2-enzyme sensor electrode may include a ketone-specific electrode. In the example of FIG. 3, the analyte electrode 302 may be used to measure a level of a first analyte (e.g., glucose). The analyte electrode 304 may be used to measure a level of a second analyte (e.g., lactate). The reference electrode 306 may have a known electrical potential that may serve as a reference potential when measuring or determining the electrical potential of the other electrodes.

During sensing, the deposited selective molecular recognition elements may be utilized to convert a respective analyte to an intermediary product (e.g., hydrogen peroxide), which is then oxidized at the surface of the electrodes. The resulting current flow, which is measured by the potentiostat of the sensor electronics module 116 or an ammeter in communication with the potentiostat, is proportional to the analyte concentration. Examples of suitable enzymes include glucose oxidase for sensing glucose species, lactate oxidase for sensing lactate species, urate oxidase for sensing urate species, glutamate oxidase for glutamate species, and the like. In addition to enzymes, the active surfaces may further include immobilized redox mediators/electron-transfer (e.g., relays) (not shown), which are small electroactive molecules for shuttling electrons between the enzymes and the electrodes. In other embodiments, active surfaces may further include enzyme co-factors, which are compounds used by the enzyme to convert a substrate to a product (e.g., FAD, FMN, NAD). In some embodiments, the selective molecular recognition elements are immobilized exclusively over the skive regions of electrodes to minimize or avoid cross-talk between different analytes. In other words, an area of active surface over each of the electrodes may be less than a geometric surface area of the respective working electrode. Maintaining a potential bias at the electrodes may facilitate a near-zero peroxide efflux from the skive region with active consumption of the hydrogen peroxide intermediary. In other embodiments, a current, voltage, open circuit potential, or frequency-dependent impedance value may be measured directly from the interaction of selective molecular recognition elements with the analyte of interest in lieu of conversion to an electroactive intermediary product.

The continuous analyte monitoring system 104 may use one or more of the signal streams from the analyte electrode 302 and/or the analyte electrode 304 to determine if one of the analyte electrodes 302 or 304 are defective. In some embodiments, the continuous analyte monitoring system 104 generates a first analyte signal stream using the analyte electrode 302 and a second analyte signal stream using the analyte electrode 304. For example, the analyte electrodes 302 and 304 may generate current flows when particular analytes interact with the surfaces of the analyte electrodes 302 and 304. The potentiostat of the sensor electronics module 116 may measure and report these current flows to the processor of the processor module 214. The processor may then generate the analyte signal streams that represent the measured current flows. The continuous analyte monitoring system 104 then determines a signal-to-noise ratio (SNR) for each of the analyte signal streams. If the SNR for the second analyte signal stream exceeds the SNR for the first analyte signal stream (e.g., exceeds the SNR for the first analyte signal stream by an amount that exceeds a threshold), then the continuous analyte monitoring system 104 may determine that the analyte electrode 302 is defective. The continuous analyte monitoring system 104 may then communicate a message or alert (e.g., to a display device 106, 108, 110, or 112) that the analyte electrode 302 should be replaced. It may also invoke a mitigation (e.g., activating another analyte sensor) and attempt to rectify said erosion of the SNR. In certain embodiments, multiple electrodes (e.g., identical and independent electrodes) may form a redundant sensing array. If a sensor in the array is determined to be defective, the sensor may be deactivated or otherwise excluded from population-level statistics (e.g., signal mean).

In some embodiments, the continuous analyte monitoring system 104 determines the SNR using multiple data streams (e.g., orthogonal data streams such as an analyte sensor signal stream and a temperature sensor signal stream). If both data streams decrease simultaneously, it could indicate that a sensor was pulled out.

If the analyte electrode 302 cannot be replaced immediately, the continuous analyte monitoring system 104 may provide temporary workarounds or solutions. For example, the continuous analyte monitoring system 104 may increase the sampling rate or frequency for the analyte electrode 302 (which may also be referred to as “oversampling”). By increasing the number of samples over a period of time, the continuous analyte monitoring system 104 may improve the SNR of the first analyte signal stream. After the analyte electrode 302 has been replaced, the continuous analyte monitoring system 104 may reduce the sampling rate or frequency.

The continuous analyte monitoring system 104 may determine the SNR for a signal stream in any suitable manner. For example, the continuous analyte monitoring system 104 may determine the SNR as a ratio of the power in an analyte signal stream to the power of the signal noise. The power may be determined as the square of a detected voltage or current of the analyte signal stream or the signal noise. The continuous analyte monitoring system 104 may determine SNR over any suitable time period. For example, the continuous analyte monitoring system 104 may determine an instantaneous SNR for the analyte signal stream at peak values (e.g., when the analyte signal stream has maximum power). As another example, the continuous analyte monitoring system 104 may determine an SNR for the analyte signal stream using an average power of the analyte signal stream over a period of time.

In certain embodiments, the continuous analyte monitoring system 104 generates a reference analyte signal stream using the analyte electrode 302. For example, the continuous analyte monitoring system 104 may generate the reference analyte signal stream shortly after the analyte electrode 302 is positioned on the user 102. The continuous analyte monitoring system 104 may determine a SNR of the reference analyte signal stream. The continuous analyte monitoring system 104 may then generate a second analyte signal stream using the analyte electrode 302. For example, the continuous analyte monitoring system 104 may generate the second analyte signal stream after the analyte electrode 302 has been positioned on the user 102 for a period of time (e.g., several hours, a day or more, etc.). The continuous analyte monitoring system 104 may determine a SNR of the second analyte signal stream. If the SNR for the reference analyte signal stream exceeds the SNR for the second analyte signal stream (e.g., exceeds the SNR for the second analyte signal stream by an amount exceeding a threshold), then the continuous analyte monitoring system 104 may determine that the analyte electrode 302 is defective. The continuous analyte monitoring system 104 may then communicate a message or alert (e.g., to a display device 106, 108, 110, or 112) that the analyte electrode 302 should be replaced.

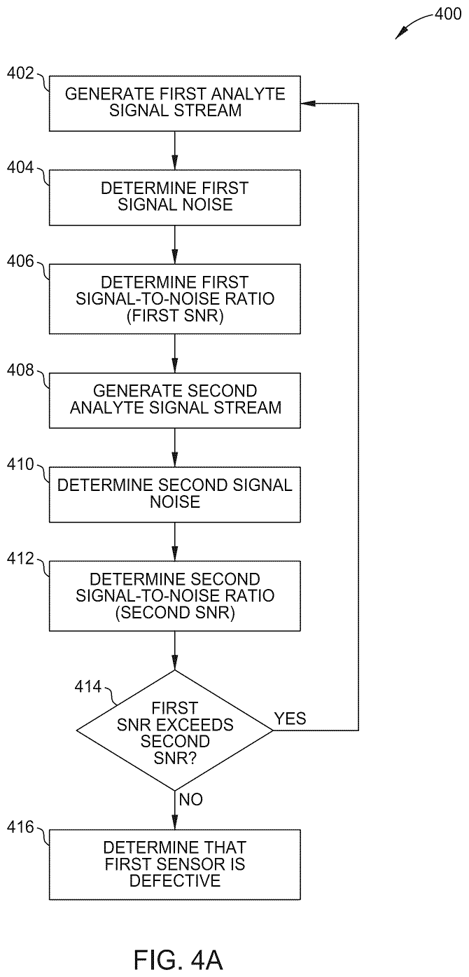

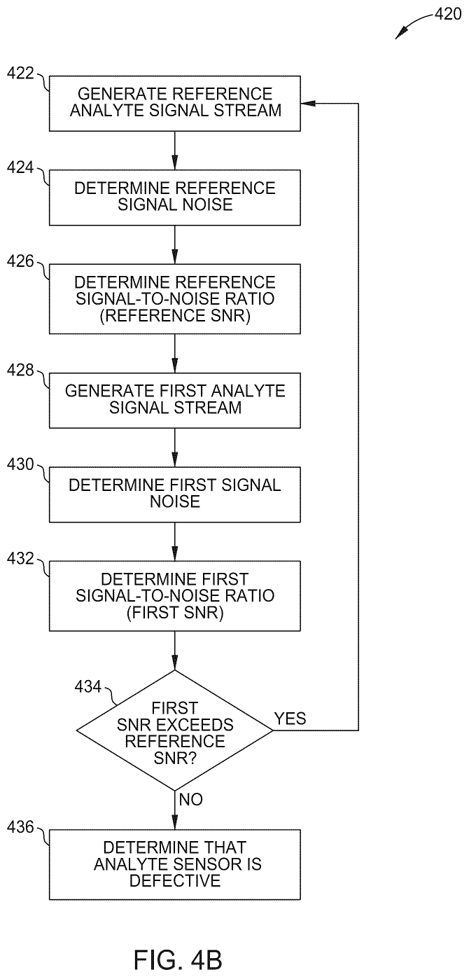

FIG. 4A illustrates a flow diagram of an example method 400 for determining whether an analyte sensor in the continuous analyte monitoring system 104 is defective, in accordance with certain aspects of the present disclosure. In particular embodiments, the continuous analyte monitoring system 104 performs the method 400.

At block 402, the continuous analyte monitoring system 104 begins by generating a first analyte signal stream. The continuous analyte monitoring system 104 may use a first analyte electrode (e.g., the analyte electrode 302) that may be part of a first analyte sensor to continuously or non-continuously monitor a first analyte of a patient after the first analyte electrode is positioned on the patient to generate an electric current corresponding to the level of the first analyte in the patient's body. The electric current may form a signal that changes over time, which forms the first analyte signal stream. The level of the electric current may indicate to the continuous analyte monitoring system 104 a level (e.g., concentration) of the first analyte. The first analyte may include lactate, though other analytes are also contemplated.

At block 404, the continuous analyte monitoring system 104 determines a first signal noise present in the first analyte signal stream. For example, the continuous analyte monitoring system 104 may perform filtering operations on the first analyte signal stream to determine the first signal noise present in the first analyte signal stream. In some embodiments, the continuous analyte monitoring system 104 determines the first signal noise as the continuous analyte monitoring system 104 generates the first analyte signal stream. For example, the continuous analyte monitoring system 104 may determine the first signal noise simultaneously with generating the first analyte signal stream. After the first analyte signal stream is generated and the first signal noise is determined, the continuous analyte monitoring system 104 determines a first SNR at block 406. The first SNR may be determined by comparing a peak amplitude of the first analyte signal stream to the level of the first signal noise (e.g., by determining a ratio of the power of the first analyte signal stream at its peak amplitude to the power of the first signal noise). Alternatively, a continuous first SNR may be determined by continually comparing the amplitude of the first analyte signal stream to the first signal noise.

At block 408, the continuous analyte monitoring system 104 generates a second analyte signal stream. The continuous analyte monitoring system 104 may use a second analyte electrode (e.g., analyte electrode 304) that may be part of a same sensor or a second analyte sensor to continuously or non-continuously monitor a second analyte of the patient after the second analyte electrode is positioned on the patient to produce an electric current corresponding to the level of the second analyte in the patient's body. The electric current may form a signal that changes over time, which forms the second analyte signal stream. The level of the electric current may indicate to the continuous analyte monitoring system 104 a level (e.g., concentration) of the second analyte. The second analyte may include glucose, though other analytes are also contemplated.

At block 410, the continuous analyte monitoring system 104 determines a second signal noise present in the second analyte signal stream. For example, the continuous analyte monitoring system 104 may perform filtering operations on the second analyte signal stream to determine the second signal noise present in the second analyte signal stream. In some embodiments, the continuous analyte monitoring system 104 determines the second signal noise as the continuous analyte monitoring system 104 generates the second analyte signal stream. For example, the continuous analyte monitoring system 104 may determine the second signal noise simultaneously with generating the second analyte signal stream. After the second analyte signal stream is generated and the second signal noise is determined, the continuous analyte monitoring system 104 determines a second SNR at block 412. The second SNR may be determined by comparing a peak amplitude of the second analyte signal stream to the level of the second signal noise. Alternatively, a continuous second SNR may be determined by continually comparing the amplitude of the second analyte signal stream to the second signal noise.

Whether one or more sensors are defective may be determined based on a comparison of the first SNR with the second SNR. For example, at block 414, the continuous analyte monitoring system 104 compares the first SNR with the second SNR to determine whether the first SNR exceeds the second SNR. If the first SNR exceeds the second SNR, then the continuous analyte monitoring system 104 may determine that the first analyte sensor is not defective and return to block 402. However, if the first SNR does not exceed (e.g., is less than) the second SNR, the continuous analyte monitoring system 104 determines that the first analyte sensor is defective (e.g., damaged or non-responsive) in block 416. The continuous analyte monitoring system 104 may communicate an alert to replace the defective sensor. The continuous analyte monitoring system 104 may also request a user action in response to determining that the first analyte sensor is defective in block 416. For example, the continuous analyte monitoring system 104 may display a prompt on the display device 107 requesting that the user replace the first analyte sensor.

In some embodiments, the continuous analyte monitoring system 104 may determine that the first analyte sensor is defective if the first SNR falls below the second SNR by an amount greater than a threshold or if the second SNR exceeds the first SNR by an amount greater than the threshold. Thus, if the first SNR falls below the second SNR but by an amount less than the threshold, then the continuous analyte monitoring system 104 may determine that the first analyte sensor is not defective and return to block 402. If the first SNR falls below the second SNR by an amount greater than the threshold, then the continuous analyte monitoring system 104 may determine that the first analyte sensor is defective.

As another example, the continuous analyte monitoring system 104 may determine a noise figure (NF) as a ratio of the first SNR and the second SNR, or vice versa. If the NF falls below or exceeds a threshold value, then the continuous analyte monitoring system 104 may determine that the first analyte sensor is defective. The NF can also be measured over time and compared to a reference value. A declining NF over time could indicate that the first analyte sensor is attaining equilibrium with its surroundings and could indirectly indicate that improved accuracy can be obtained. An increasing NF could indicate that the first analyte sensor is experiencing an end-of-life event (e.g., fibrous encapsulation) or an external perturbation (e.g., motion artifact, pressure-induced signal attenuation, etc.).

In certain embodiments, the SNR of the signal stream from a first of the analyte sensors in a cohort of three or more analyte sensors may be compared with the mean SNR of the signal streams from the other two or more analyte sensors to determine if the first analyte sensor is defective. For example, the first analyte sensor may be defective if the SNR from its signal stream falls below the mean SNR. As another example, a leave-one-out cross-validation approach may be used so that each analyte sensor in a cohort of three or more analyte sensors is evaluated against the other two or more analyte sensors. The SNR value of the analyte sensor under examination may be compared against a mean or median SNR value of the other analyte sensors. The analyte sensor under examination may be determined to be defective if its SNR value falls beneath the mean or median SNR value (e.g., by an amount greater than a predefined variance). This cross-validation process may repeated for every analyte sensor in the cohort so that each sensor is evaluated in the same manner. This process may also be referred to as voting or polling.

Alternatively, a defective sensor may be detected in a single sensor configuration using an example method 420 as shown in FIG. 4B. FIG. 4B illustrates a flow diagram of an example method 420 for determining whether an analyte sensor in the continuous analyte monitoring system 104 is defective, in accordance with certain aspects of the present disclosure. In certain embodiments, the continuous analyte monitoring system 104 performs the method 420.

At block 422, the continuous analyte monitoring system 104 begins by generating a reference analyte signal stream. The continuous analyte monitoring system 104 may use an analyte sensor (e.g., the analyte electrode 302) to continuously or non-continuously monitor an analyte (e.g., glucose or lactate) of a patient after the analyte sensor is positioned on the patient to produce an electric current corresponding to a level of the analyte in the patient's body. The electric current may form a signal that changes over time, which forms the reference analyte signal stream. The level of the electric current may indicate to the continuous analyte monitoring system 104 a level (e.g., concentration) of the analyte. The continuous analyte monitoring system 104 may use the analyte sensor to generate the reference analyte signal stream during a time period shortly following positioning (e.g., insertion) of the analyte sensor onto the patient's body.

At block 424, the continuous analyte monitoring system 104 determines a reference signal noise present in the reference analyte signal stream. For example, the continuous analyte monitoring system 104 may perform filtering operations on the reference analyte signal stream to determine the reference signal noise present in the reference analyte signal stream. In some embodiments, the continuous analyte monitoring system 104 determines the reference signal noise as the continuous analyte monitoring system 104 generates the reference analyte signal stream. For example, the continuous analyte monitoring system 104 may determine the reference signal noise simultaneous with generating the reference analyte signal stream. After the reference analyte signal stream is generated and the reference signal noise is determined, the continuous analyte monitoring system 104 determines a reference SNR at block 426. The reference SNR may be determined by comparing a peak amplitude of the reference analyte signal stream to the level of the reference signal noise. Alternatively, a continuous reference SNR may be determined by continually comparing the amplitude of the reference analyte signal stream to the reference signal noise.

At block 428, the continuous analyte monitoring system 104 generates a first analyte signal stream. The continuous analyte monitoring system 104 may use the analyte sensor to continuously or non-continuously monitor the analyte to generate the first analyte signal stream. The analyte sensor may produce an electric current corresponding to the level of the analyte in the patient's body. In some instances, the analyte sensors may provide an electric voltage (e.g., using ion-selective sensors) corresponding to the level of the analyte in the patient's body. The electric current may form a signal that changes over time, which forms the first analyte signal stream. The level of the electric current may indicate to the continuous analyte monitoring system 104 a level (e.g., concentration) of the analyte.

At block 430, the continuous analyte monitoring system 104 determines a first signal noise present in the first analyte signal stream. For example, the continuous analyte monitoring system 104 may perform filtering operations on the first analyte signal stream to determine the first signal noise present in the first analyte signal stream. In some embodiments, the continuous analyte monitoring system 104 determines the first signal noise as the continuous analyte monitoring system 104 generates the first analyte signal stream. For example, the continuous analyte monitoring system 104 may determine the first signal noise simultaneous with generating the first analyte signal stream. After the first analyte signal stream is generated and the first signal noise is determined, the continuous analyte monitoring system 104 determines a first SNR at block 432. The first SNR may be determined by comparing a peak amplitude of the first analyte signal stream to the level of the first signal noise. Alternatively, a continuous first SNR may be determined by continually comparing the amplitude of the first analyte signal stream to the first signal noise.

In certain embodiments, the SNR may be calculated from an impedance value corresponding to the reference analyte signal stream and may be obtained from a stimulus at a single frequency or continuum of frequencies. The SNR may constitute a total complex impedance value, a real component of the total complex impedance value, an imaginary component of the total complex impedance value, the amplitude component of the impedance value, or the phase component of the impedance value.

At block 434, the continuous analyte monitoring system 104 compares the reference SNR with the first SNR and determines whether the first SNR exceeds the reference SNR. If the first SNR exceeds the reference SNR, then the continuous analyte monitoring system 104 may determine that the analyte sensor is not defective and return to block 422. However, if the first SNR does not exceed (e.g., is less than) the reference SNR, the continuous analyte monitoring system 104 determines that the analyte sensor is defective (e.g., damaged or non-responsive) in block 436. The continuous analyte monitoring system 104 may communicate an alert in response to determining that the sensor is defective. The continuous analyte monitoring system 104 may also request a user action in response to determining that the sensor is defective in block 436. For example, the continuous analyte monitoring system 104 may display a prompt on the display device 107 requesting that the user replace the sensor of the continuous analyte monitoring system 104.

In some embodiments, the continuous analyte monitoring system 104 may determine that the analyte sensor is defective if the first SNR falls below the reference SNR by an amount greater than a threshold or does not exceed the reference SNR by an amount greater than a threshold. Thus, if the first SNR falls below the reference SNR but by an amount less than a threshold or if the first SNR exceeds the reference SNR by an amount greater than a threshold, then the continuous analyte monitoring system 104 may determine that the analyte sensor is not defective and return to block 422. If the first SNR falls below the reference SNR by an amount greater than a threshold or if the first SNR exceeds the reference SNR but by an amount less than a threshold, then the continuous analyte monitoring system 104 may determine that the analyte sensor is defective.

As another example, the continuous analyte monitoring system 104 may determine a noise figure (NF) as a ratio of the first SNR and the reference SNR, or vice versa. If the NF falls below a threshold value, then the continuous analyte monitoring system 104 may determine that the first analyte sensor is defective. The NF can also be measured over time and compared to a reference value. A declining NF over time could indicate that the first analyte sensor is attaining equilibrium with its surroundings and could indirectly indicate that improved accuracy can be obtained. An increasing NF could indicate that the first analyte sensor is experiencing an end-of-life event (e.g., fibrous encapsulation) or an external perturbation (e.g., motion artifact, pressure-induced signal attenuation, etc.).

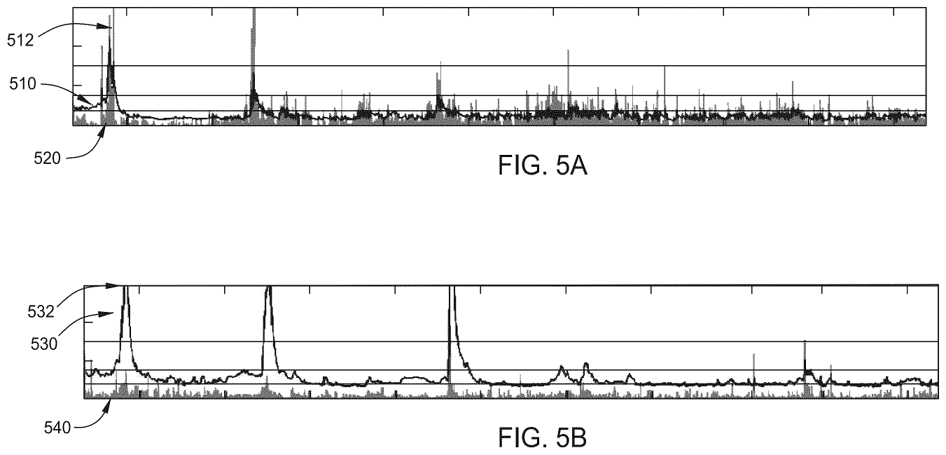

FIGS. 5A and 5B illustrate analyte signal streams and signal noise over a period of time. FIG. 5A illustrates an analyte signal stream 510 from a defective sensor. The analyte signal stream 510 may include noise 520 and an analyte signal 512. FIG. 5B illustrates an analyte signal stream 530 from a non-defective sensor. The analyte signal stream 530 includes noise 540 and an analyte signal 532. The analyte signal 512 has a peak that is less than the peak of the noise 520. By contrast, the analyte signal 532 has a peak that is higher than the peak of the noise 540. As a result, the SNR of the analyte signal stream 510 may be significantly less than the SNR of the analyte signal stream 530. Thus, if the continuous analyte monitoring system 104 compares the SNR of the analyte signal stream 510 to the SNR of the analyte signal stream 530 or to a reference SNR, the continuous analyte monitoring system 104 may determine that the analyte sensor that produced the analyte signal stream 510 may be defective.

The methods disclosed herein include one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a c c, b-b, b-b-b, b-b-c, c-c, and c-c-c or any other ordering of a, b, and c).

The term “calibration,” as used herein, is a broad term, and is used in its ordinary sense, and refers without limitation to a process of determining the relationship between raw sensor data to clinically meaningful units.

The term “calibrated data,” as used herein, is a broad term, and is used in its ordinary sense, and refers without limitation to data that has been transformed from its raw state to another state using a function or series of functions, lookup table, etc., to provide a clinically meaningful value to a user.

The term “continuous,” as used herein, is a broad term, and is used in its ordinary sense, and can mean continuous, semi-continuous, continual, periodic, intermittent, episodic, regular, etc.

The terms “continuous analyte sensor,” “continuous multi-analyte sensor,” “continuous glucose sensor,” and “continuous lactate sensor,” as used herein, are broad terms, and are used in their ordinary sense, and refer without limitation to a device that continuously measures a concentration of an analyte or calibrates the device (e.g., by continuously adjusting or determining the sensor's sensitivity and background), for example, at time intervals ranging from fractions of a second up to, e.g., 1, 2, or 5 minutes, or longer.

The terms “sensitivity” or “sensor sensitivity,” as used herein, are broad terms, and are used in their ordinary sense, and refer without limitation to an amount of signal produced by a certain concentration of a measured analyte, or a measured species (e.g., H₂O₂) associated with a measured analyte (e.g., glucose or lactate). For example, a sensor may have a sensitivity of from about 1 to about 300 picoAmps of current for every 1 mg/dL of glucose analyte. For measurement of ions, a sensor may have sensitivity close to the Nernstian limit, about 59.13 mV for every logarithmic (e.g., log base 10) increase of ion concentration at room temperature.

The term “sensor data,” as used herein, is a broad term, and is used in its ordinary sense, and refers without limitation to any data associated with a sensor, such as a continuous analyte or continuous multi-analyte sensor. Sensor data includes a raw data stream, or simply data stream, of analog or digital signal directly related to a measured analyte from an analyte sensor (or other signal generated from another sensor), as well as calibrated or filtered raw data. The terms “sensor data point” and “data point” refer generally to a digital representation of sensor data at a particular time. The terms broadly encompass a plurality of time spaced data points from a sensor, such as a continuous analyte sensor, which includes individual measurements taken at time intervals ranging from fractions of a second up to, e.g., 1, 2, or 5 minutes or longer. In another example, the sensor data includes an integrated digital value representative of one or more data points averaged over a time period. Sensor data may include calibrated data, smoothed data, filtered data, transformed data, or any other data associated with a sensor.

The term “sensor electronics,” as used herein, is a broad term, and is used in its ordinary sense, and refers without limitation to components, e.g., hardware or software, of a device configured to process sensor data and can include an electrochemical analog front end, a potentiostat, a galvanostat, an impedance analyzer, a frequency response analyzer, an analog-to-digital converter, a microprocessor, a wireless radio, and/or a battery.

Although certain embodiments herein are described with reference to management of diabetes, diabetes management is only an example of one application for which the present systems and methods may be utilized. The systems and methods described herein can also be used for managing one or more other diseases or conditions, which may or may not include diabetes. For example, the systems and methods described herein can be utilized for managing kidney disease, liver disease, and other types of diseases or conditions.

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but is to be accorded the full scope consistent with the language of the claims, wherein reference to an element in the singular is not intended to mean “one and only one” unless specifically so stated, but rather “one or more.” Unless specifically stated otherwise, the term “some” refers to one or more. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112 (f) unless the element is expressly recited using the phrase “means for” or, in the case of a method claim, the element is recited using the phrase “step for.”

While various examples of the invention have been described above, it should be understood that they have been presented by way of example only, and not by way of limitation. Likewise, the various diagrams may depict an example architectural or other configuration for the disclosure, which is done to aid in understanding the features and functionality that can be included in the disclosure. The disclosure is not restricted to the illustrated example architectures or configurations, but can be implemented using a variety of alternative architectures and configurations. Additionally, although the disclosure is described above in terms of various example examples and aspects, it should be understood that the various features and functionality described in one or more of the individual examples are not limited in their applicability to the particular example with which they are described. They instead can be applied, alone or in some combination, to one or more of the other examples of the disclosure, whether or not such examples are described, and whether or not such features are presented as being a part of a described example. Thus the breadth and scope of the present disclosure should not be limited by any of the above-described example examples.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘including’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide example instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular example of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

The term “including as used herein is synonymous with “including,” “containing,” or “characterized by” and is inclusive or open-ended and does not exclude additional, unrecited elements or method steps.

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific examples and examples described herein, but rather to also cover all modification and alternatives coming with the true scope and spirit of the invention.

What is claimed is:

1. An analyte sensor system comprising:
a sensor;
a memory; and
a processor communicatively coupled to the memory, the processor configured to:
generate, using the sensor, a reference analyte signal stream;
determine a reference signal noise in the reference analyte signal stream;
compare the reference analyte signal stream to the reference signal noise to determine a reference signal-to-noise ratio;
generate, using the sensor, an analyte signal stream;
determine a signal noise in the analyte signal stream;
determine a signal-to-noise ratio based on the analyte signal stream and the signal noise; and
in response to determining that the signal-to-noise ratio is less than the reference signal-to-noise ratio, determine that the sensor is defective.

2. The analyte sensor system of claim 1, wherein the reference analyte signal stream indicates at least one of a lactate level, a ketone level, a creatinine level, a uric acid level, an ethanol level, a sodium level, a potassium level, or a calcium level.

3. The analyte sensor system of claim 1, wherein generating the reference analyte signal stream occurs after the sensor is positioned on a body of a user.

4. The analyte sensor system of claim 1, wherein comparing the reference analyte signal stream to the reference signal noise comprises comparing a peak of the reference analyte signal stream to the reference signal noise.

5. The analyte sensor system of claim 1 wherein determining the reference signal noise occurs while generating the reference analyte signal stream.

6. The analyte sensor system of claim 1, wherein comparing the analyte signal stream to the signal noise comprises comparing a peak of the analyte signal stream to the signal noise.

7. The analyte sensor system of claim 1, wherein determining the signal noise occurs while generating the analyte signal stream.

8. The analyte sensor system of claim 1, wherein the processor is further configured to communicate at least one of an alert, a notification, or an alarm in response to determining that the sensor is defective.

9. The analyte sensor system of claim 1, wherein the processor is further configured to request a user action in response to determining that the sensor is defective.

10. An analyte sensor system, comprising:
a first sensor;
a second sensor;
a memory; and
a processor communicatively coupled to the memory, the processor configured to:
generate, using the first sensor, a first analyte signal stream indicating a level of a first analyte;
determine a first signal noise in the first analyte signal stream;
determine a first signal-to-noise ratio based on the first analyte signal stream and the first signal noise;
generate, using the second sensor, a second analyte signal stream indicating a level of a second analyte;
determine a second signal noise in the second analyte signal stream;
determine a second signal-to-noise ratio based on the second analyte signal stream and the second signal noise; and
in response to determining that the second signal-to-noise ratio exceeds the first signal-to-noise ratio, determine that the first sensor is defective.

11. The analyte sensor system of claim 10, wherein determining the first signal-to-noise ratio comprises comparing a peak of the first analyte signal stream to the first signal noise.

12. The analyte sensor system of claim 10, wherein determining the first signal noise occurs while generating the first analyte signal stream.

13. The analyte sensor system of claim 10, wherein generating the first analyte signal stream and the second analyte signal stream occurs after the first sensor is positioned on a body of a user.

14. The analyte sensor system of claim 10, wherein generating the first analyte signal stream occurs while generating the second analyte signal stream.

15. The analyte sensor system of claim 10, wherein the first analyte comprises lactate and the second analyte comprises glucose.

16. A method, comprising:
generating, using a sensor, a reference analyte signal stream;
determining a reference signal noise in the reference analyte signal stream;
comparing the reference analyte signal stream to the reference signal noise to determine a reference signal-to-noise ratio;
generating, using the sensor, an analyte signal stream;
determining a signal noise in the analyte signal stream;
determining a signal-to-noise ratio based on the analyte signal stream and the signal noise; and
in response to determining that the reference signal-to-noise ratio exceeds the signal-to-noise ratio, determining that the sensor is defective.

17. The method of claim 16, wherein generating the reference analyte signal stream occurs after the sensor is positioned on a body of a user.

18. The method of claim 16, wherein determining the reference signal-to-noise ratio comprises comparing a peak of the reference analyte signal stream to the reference signal noise.

19. The method of claim 16, further comprising requesting a user action in response to determining that the sensor is defective.

20. The method of claim 16, wherein the reference analyte signal stream indicates a lactate level.
